(19) United States
(12) Patent Application Publication
(10) Pub. No.: US 2022/0096367 A1
(43) Pub. Date: Mar. 31, 2022

(54) ORAL THIN FILM

(71) Applicant: LTS LOHMANN THERAPIE-SYSTEME AG, Andemach (DE)

(72) Inventors: Marius BAUER, Andernach (DE); Michael LINN, Waldböckelheim (DE); Marco EMGENBROICH, Rheinbach (DE); Christoph SCHMITZ, Rheinbrohl (DE); Markus MÜLLER, Troisdorf (DE)

(21) Appl. No.: 17/421,265

(22) PCT Filed: Jan. 10, 2020
(86) PCT No.: PCT/EP2020/050569

§ 371 Date: Jul. 7, 2021

(30) Foreign Application Priority Data
Jan. 10, 2019 (DE) 10 2019 100 483.7

(51) Int. Cl. A61K 9/00; A61K 31/352; A61K 31/355; A61K 47/14

(52) U.S. Cl. CPC A61K 9/006; A61K 47/14; A61K 31/355; A61K 31/352

(57) ABSTRACT

The present invention additionally relates to a dosage form for an active substance, selected from the group of cannabinoids, for dissolving in the oral cavity, comprising a first film layer and a second film layer, arranged over the first film layer, wherein the composition of the first film layer can be identical to that of the second film layer and comprises a water soluble polymer, the first and second film layer being connected to each other via the overlapping edges thereof, forming at least one cavity, and the cavity being filled with an active substance selected from the group of cannabinoids.

The present invention relates to an oral thin film for the administration of active substances from the group of cannabinoids, as well as to a delivery form with a cavity for these active substances, to methods for its production, and to its use as a medicament.

The oral administration of active substances from the group of cannabinoids in the form of capsules, tablets, pills, other solid oral dosage forms or in the form of liquid preparations to be administered orally is disadvantageous for several reasons.

On the one hand, the active substance is absorbed in the gastrointestinal tract. This delays the time of onset of action, which is contrary to a rapid onset of action. On the other hand, the active substances from the group of cannabinoids are at least partially degraded and/or inactivated during the gastrointestinal passage under the influence of acids or enzymes, so that only a part of the ingested dose can actually act as active substance. In addition, after oral administration, a significant part of the active substance is already metabolised during the first liver passage (“first-pass” effect).

The problems described above can be overcome by administering an active substance from the group of cannabinoids by means of an oral thin film. Oral thin films, also called transmucosal delivery systems, are thin polymer-based, active-substance-containing films which, when applied to a mucous membrane, especially the oral mucosa, deliver the active substance directly into it. These delivery systems have the advantage that most of the active substance is absorbed through the mucosa, thus avoiding the “first-pass metabolism” that has to be taken into account with the conventional delivery form of an active substance in tablet form.

The administration of active substances from the group of cannabinoids in the form of oral thin films is known from the prior art.

For example, WO 03/105800 A2 discloses an oral thin film for the administration of cannabinoids in which the active substance is embedded in a hydrophilic, water-soluble matrix.

CA 2922959 A1 discloses an oral thin film for the administration of cannabinoids, wherein the active substances are in the form of nanomicelles, comprising the active substance in aqueous solution, in an outer film-forming polymer.

In addition, freely and commercially available cannabis strips are known from the USA, wherein a THC extract is embedded in a hydrophilic polymer matrix alongside flavourings.

However, the prior art dosage forms of cannabinoids in the form of oral thin films or strips have the disadvantage that the active substances from the group of cannabinoids are relatively susceptible to oxidation and thus relatively unstable when they are coarsely dispersed in a water-soluble polymer. Especially, tetrahydrocannabinol (THC), when embedded in a hydrophilic matrix, is present in waxy/resinous form as a separate phase, which is especially disadvantageous with regard to oxidative degradation of the active substance and the stability of the oral thin film with regard to phase separation. Due to the sensitivity of the active substances from the group of cannabinoids to oxidative degradation, the oral thin films known from the prior art are disadvantageous, especially with regard to their storage stability. In addition, the dosage forms in the form of cannabis strips have the disadvantage that these strips are relatively thick and thus inflexible, which impairs the wearing comfort in the oral cavity.

The aim of the present invention is to overcome the above-mentioned disadvantages of the prior art. Especially, the aim of the present invention is to provide an oral thin film or a transmucosal delivery system in which the active substances from the group of cannabinoids are chemically stable, i.e. especially protected from oxidative degradation. Especially, the oral thin film should be able to be stored over a longer period of time without the active substance from the group of cannabinoids being significantly degraded, especially oxidatively. In addition, the oral thin film should be easy and inexpensive to produce.

The above aim is addressed by an oral thin film or transmucosal delivery system according to claim 1, which comprises an outer hydrophilic phase containing at least one hydrophilic polymer and an inner hydrophobic phase containing at least one hydrophobic substance and at least one pharmaceutically active substance selected from the group of cannabinoids, the oral thin film additionally comprising at least one emulsifier and/or vitamin E and/or a pharmaceutically acceptable derivative of vitamin E.

Such a system has the advantage that the active substance from the group of cannabinoids is present in a hydrophobic environment that is stabilised in the hydrophilic phase, which largely prevents phase separation in the oral thin film. In addition, the active substance from the group of cannabinoids, when present in a hydrophobic environment, is significantly better stabilised against oxidative degradation. In addition, the active substance from the group of cannabinoids is present in solution in a hydrophobic environment, which can also have a positive effect on absorption. Dissolving the active substance from the group of cannabinoids in a hydrophobic substance also significantly facilitates the processing of the active substance and the production of the oral thin film.

The oral thin film according to the invention is preferably present as an oil-in-water emulsion. This oil-in-water emulsion is preferably stabilised by the at least one emulsifier.

It has also been shown that vitamin E and/or a pharmaceutically acceptable derivative of vitamin E, preferably in an amount of at least about 1% by weight, wherein the vitamin E and/or the pharmaceutically acceptable derivative of vitamin E itself has no emulsifying properties but serves as an additional solvent for the at least one active substance from the group of cannabinoids, can stabilise the inner hydrophobic phase containing the at least one active substance from the group of cannabinoids also in the hydrophilic phase. Vitamin E and/or a pharmaceutically acceptable derivative of vitamin E, preferably in an amount of at least about 1% by weight, can thus be used in addition to or instead of the at least one emulsifier.

Due to the hydrophobicity of vitamin E and/or the pharmaceutically acceptable derivative of vitamin E, vitamin E and/or the pharmaceutically acceptable derivative of vitamin E together with the at least one hydrophobic substance and the at least one active substance from the group of cannabinoids is preferably present in the inner hydrophobic phase. The term “hydrophobic substance” is expressly not intended to include the hydrophobic substance vitamin E and/or a pharmaceutically acceptable derivative of vitamin E.

Vitamin E comprises the chemical compounds α-tocopherol, β-tocopherol, γ-tocopherol, δ-tocopherol and α-tocotrienol, β-tocotrienol, γ-tocotrienol and δ-tocotrienol.

α-tocopherol acetate is especially preferred as pharmaceutically acceptable derivative of vitamin E.

In a preferred embodiment of the oral thin film according to the invention, the active substance from the group of cannabinoids is present substantially in the inner hydrophobic phase of the system. The term “substantially” shall be understood to mean that the active substance from the group of cannabinoids is present in the inner hydrophobic phase of the oral thin film to an extent of more than about 80% by weight, preferably more than about 85% by weight and especially preferably more than about 90% by weight, very especially preferably more than about 95% by weight and even more preferably more than about 99% by weight, in relation to the total amount of active substance from the group of cannabinoids in the oral thin film.

This has the advantage that the active substance from the group of cannabinoids is substantially dissolved in the inner phase and is thus chemically and/or physically more stable. If less than 80% by weight of the active substance from the group of cannabinoids is present in the inner hydrophobic phase, this has the disadvantage that too much of the active substance from the group of cannabinoids is present in the outer hydrophilic phase, which favours oxidative degradation of the active substance.

The term cannabinoids stands for a collective term for terpenoid ingredients which have 21 carbon atoms and which can be isolated from cannabis species, mainly benzopyran derivatives, and their (semi-)synthetic derivatives. More than 70 naturally occurring cannabinoids are known, some of which have psychotropic effects and others pharmacological effects.

A non-exhaustive list of examples of cannabinoids includes: cannabichromanone, cannabichromene, cannabicoumaronone, cannabicyclol, cannabidiol, cannabidivarin, cannabidivaric acid, cannabifuran, cannabinodiol, cannabinol, cannabinolic acid, cannabitriol, cannabivarichromene, cannabivarin, Δ8-tetrahydrocannabinol, Δ9-tetrahydrocannabinol. The active substances from the group of cannabinoids may be of natural, semi-synthetic or synthetic origin.

As a synthetically produced cannabinoid, R-(6a, 10a)-Δ9-tetrahydrocannabinol is also suitable for administration in the oral thin film according to the invention.

Cannabis extracts and Cannabis oils, especially extracts and oils of Cannabis sativa or Cannabis indica, may also be considered. Cannabis extracts or oils contain, inter alia, tetrahydrocannabinol (predominantly Δ9-tetrahydrocannabinol, in a smaller proportion Δ8-tetrahydro-cannabinol), cannabidiol, cannabinol and cannabichromene as pharmacologically active ingredients. Especially preferably, the oral thin film according to the invention comprises at least one pharmaceutically active substance selected from the group of cannabinoids tetrahydrocannabinol (THC), preferably Δ8-tetrahydrocannabinol, Δ9-tetrahydrocannabinol or R-(6a, 10a)-Δ9-tetrahydrocannabinol, cannabinol, cannabidiol and/or cannabichromene.

The oral thin film according to the invention is additionally preferably characterised in that the amount of the at least one pharmaceutically active substance selected from the group of cannabinoids is about 1 to 30% by weight, preferably about 2 to 25% by weight, especially preferably about 5 to 20% by weight and even more preferably about 8 to 12% by weight, in relation to the total weight of the oral thin film.

The hydrophilic polymer in the outer hydrophilic phase is a polymer that contains polar or charged groups. These groups can be non-ionic, anionic, cationic or zwitterionic. Hydrophilic polymers are usually soluble in water.

Especially preferably, the hydrophilic polymer in the outer hydrophilic phase is a hydrophilic and water-soluble polymer. Water-soluble polymers comprise chemically very different, natural or synthetic polymers, the common feature of which is their solubility in water or aqueous media. The prerequisite for this is that these polymers have a sufficient number of hydrophilic groups for water solubility and are not cross-linked.

The hydrophilic polymer of the outer hydrophilic phase of the oral thin film according to the invention preferably comprises a hydrophilic polymer selected from the group consisting of starch and starch derivatives, dextran, cellulose and cellulose derivatives, such as carboxymethyl cellulose, hydroxypropyl cellulose, hydroxyethyl cellulose, hydroxypropylmethyl cellulose, hydroxypropylethyl cellulose, sodium carboxymethyl cellulose, ethyl or propyl cellulose, polyacrylic acid, polyacrylate, polyvinylpyrrolidone, polyethylene glycol/polyvinyl alcohol copolymer, polyvinyl alcohol, polyethylene oxide polymers, polyethylene oxide/polyethylene glycol copolymers, polyacrylamide, polyethylene glycol, gelatine, collagen, alginate, pectin, pullulan, tragacanth, chitosan, alginic acid, arabinogalactan, galactomannan, agar-agar, agarose, carrageenan, shellac, natural gums and/or copolymers thereof.

In an especially preferred embodiment, the at least one hydrophilic polymer comprises pullulan, polyvinyl alcohol, polyvinylpyrrolidone, a cellulose derivative, especially hydroxypropylmethyl cellulose and/or copolymers thereof. The use of polyvinyl alcohol is especially preferred.

These hydrophilic polymers have the advantage that when dried they form a thin stable film which, when applied to the mucosa, dissolves in a pharmaceutically acceptable period of time and thus releases the active substance, which has the advantage of relatively rapid availability of the active substance as well as residue-free administration of the active substance.

Polyvinyl alcohol especially has the advantage that polyvinyl alcohol itself has emulsifying properties and can thus stabilise the oral thin film with regard to phase separation.

The inner hydrophobic phase of the oral thin film according to the invention comprises at least one hydrophobic substance.

A hydrophobic substance is understood to be a substance of which the logP value is greater than about 1, preferably greater than about 1.5, especially preferably greater than about 2.

The n-octanol/water partition coefficient K_{ow} (notations such as octanol/water partition coefficient are also common and correct) is a dimensionless partition coefficient known to a person skilled in the art, which indicates the ratio of the concentrations of a chemical in a two-phase system of n-octanol and water and is thus a measure of the hydrophobicity or hydrophilicity of a substance. The logP value is the decadic logarithm of the n-octanol-water partition coefficient K_{ow}. The following applies:

$${K}_{o w} = P = \frac{{c}_{o}^{Si}}{{c}_{w}^{Si}} and log P = log \frac{{c}_{o}^{Si}}{{c}_{w}^{Si}} = log {c}_{o}^{S i} - {c}_{w}^{S i}$$

with cₒ^{si}=concentration of a chemical in the octanol-rich phase and c_{w}^{Si}=concentration of a chemical in the water-rich phase.

K_{ow} is greater than one if a substance is more soluble in fat-like solvents such as n-octanol, and less than one if it is more soluble in water. Accordingly, log P is positive for lipophilic and negative for hydrophilic substances. Since the cannabinoids are hydrophobic substances, the cannabinoids preferably dissolve in a hydrophobic substance with a logP value greater than about 1, preferably greater than about 1.5, especially preferably greater than about 2.

Advantageously, the hydrophobic substance is selected from the group of pharmaceutically acceptable hydrophobic substances, such as isopropyl myristate, isopropyl palmitate, isopropyl stearate, isopropyl oleate, n-butyl stearate, n-hexyl laurate, n-decyl oleate, isooctyl stearate, isononyl stearate, isononyl isonanoate, 2-ethylhexyl palmitate, 2-ethylhexyl laurate, 2-hexyl decyl stearate, dialkyl ethers, alcohols, fatty acid triglycerides such as triglycerol esters of saturated and/or unsaturated alkanecarboxylic acids, glycerol mono- and dioleates, synthetic, semi-synthetic and natural oils such as olive oil, almond oil, avocado oil, sunflower oil, soya oil, peanut oil, rapeseed oil, palm oil, coconut oil, palm kernel oil, 2-octyldodecyl palmitate, ethyl oleate, oleyl oleate, oleyl curate, erucyl oleate and synthetic, semi-synthetic and natural mixtures of such esters, medium-chain triglycerides, paraffin oil, squalene or squalane, fatty alcohols with 6 to 18 carbon atoms in straight chains and/or acids from the group lauric, palmitic, myristic, aradidonic, oleic, linolenic and linoleic acid, methyl salicylate, tributyl citrate, triethyl citrate, eucalyptol, 1,2-propanediol and/or methyl salicylate.

Medium-chain triglycerides, especially isopropyl myristate, fatty acids, especially lauric acid, and/or mixtures thereof are especially preferred.

Especially, the term hydrophobic substance does not include vitamin E and/or a pharmaceutically acceptable derivative of vitamin E.

The oral thin film according to the invention preferably comprises at least one emulsifier. An emulsifier is a term for excipients for the production and stabilisation of emulsions which, in a narrower sense, can also be described as surface-active substances or surfactants and are usually present as oily to waxy, but also powdery substances. For the stabilisation of emulsions over a longer period of time, excipients are required which prevent or delay the segregation of the two phases oil and water to the thermodynamically stable final state until the emulsion has fulfilled its purpose. This can be achieved by emulsifiers and/or stabilisers.

Examples of emulsifiers that can be used are soaps, metal soaps, organic soaps such as ethanolamine oleates or stearates, sulphurised compounds such as sodium dodecyl sulphate, quaternary ammonium compounds, fatty alcohols such as lauryl, cetyl, stearyl, or palmityl alcohol, partial fatty acid esters of polyhydric alcohols with saturated fatty acids, such as glycerol monostearate, pentaerythritol monostearate, ethylene glycol monostearate, propylene glycol monostearate, partial fatty acid esters of polyhydric alcohols with unsaturated fatty acids, such as glycerol monooleate, glycerol dioleate, pentaerythritol monooleate, furthermore polyoxyethylene esters of fatty acids, such as polyoxyethylene stearate, polymerisation products of ethylene oxide and propylene oxide with fatty alcohols, such as fatty alcohol polyglycol ethers, or fatty acids, such as fatty acid ethoxylates, polysorbates, sorbitan esters, macrogol glycerol hydroxy stearates, lecithin, mono- or diglycerides and/or polyoxyethylene fatty acid ethers.

Emulsifiers can be characterised by the HLB value (HLB=hydrophilic-lipophilic balance=hydro-lipophilic ratio). The HLB value is a measure of the water or oil solubility of predominantly non-ionic surfactants and the stability of emulsions.

The HLB value for non-ionic surfactants can be calculated as follows

$$H L B = 20 \times ( 1 - \frac{{M}_{l}}{M} ) ,$$

where M₁ is the molar mass of the lipophilic fraction of a molecule and M is the molar mass of the whole molecule. The factor 20 is a freely chosen scaling factor. An HLB value of 1 indicates a lipophilic compound, a chemical compound with an HLB value of 20 has a high hydrophilic fraction. A value between 3 and 8 is assigned to water-in-oil emulsifiers and a value between 8 and 18 to oil-in-water emulsifiers.

In a preferred embodiment, the oral thin film is characterised in that the at least one emulsifier has an HLB value of about 2 to 18, preferably about 3 to 16. Emulsifiers with an HLB value in this range are especially well suited for stabilising the inner hydrophobic phase containing the at least one active substance from the group of cannabinoids, in the outer hydrophilic phase containing at least one hydrophilic polymer. Emulsifiers with HLB values less than 2 or greater than 18 have the disadvantage that they do not form a stable emulsion and thus lead to unstable oral thin films.

Especially preferably, the at least one emulsifier of the oral thin film according to the invention comprises lecithin, polysorbate, sorbitan esters such as polyoxyethylene (20)-sorbitan monooleate, polyoxyethylene (23)-sorbitan monolaurate, polyoxyethylene fatty acid ethers, such as polyoxyethylene (23) lauryl ether or polyoxyethylene (2) stearyl alcohol, macrogol glycerol hydroxy stearates, glycerol mono- and dioleates, and/or mixtures thereof, as they are known for example under the trade names polysorbate 80, Span 83 or 85, Kolliphor RH40,Tween 20 Tween 80, Atmos 300, Brij S2 and Brij L-23, but is not limited to these.

The oral thin film is preferably characterised in that vitamin E and/or the pharmaceutically acceptable derivative of vitamin E is present in an amount greater than about 1% by weight, preferably in an amount of about 1 to about 50% by weight, especially preferably in an amount of about 1 to about 30% by weight, very especially preferably in an amount of about 5 to about 20% by weight, in relation to the total weight of the oral thin film.

Furthermore, it is preferred that in an embodiment of the oral thin film according to the invention in which the function of vitamin E and/or a pharmaceutically acceptable derivative of vitamin E as an additional stabilising solvent as described above is not desired, nevertheless at least one radical scavenger and/or an antioxidant is present. The at least one radical scavenger and/or antioxidant is preferably present in an amount significantly less than about 1% by weight in relation to the total weight of the oral thin film. The at least one radical scavenger and/or the antioxidant is preferably contained in an amount from about 0.005 to about 0.3% by weight, and preferably from about 0.05 to about 0.25% by weight, in relation to the total weight of the oral thin film, in the oral thin film according to the invention.

This at least one radical scavenger and/or antioxidant is a chemical compound which prevents or reduces the undesired oxidation of other substances, especially the active substance, and thus counteracts ageing of the oral thin film. Especially, radical scavengers and/or antioxidants are characterised by the fact that they prevent the oxidative degradation of sensitive molecules caused by atmospheric oxygen, in this case especially of the active substance contained.

The at least one free radical scavenger is especially preferably vitamin E and/or a pharmaceutically acceptable derivative of vitamin E, butylated hydroxyanisole, ascorbyl palmitate and/or butylated hydroxytoluene.

Preferably, the at least one pharmaceutically active substance selected from the group of cannabinoids in the oral thin film according to the invention is relatively stable against degradation, especially against oxidative degradation.

It is preferred that after 2 months of storage at 25° C. and 60% relative humidity (RH) according to ICH standards, no more than 15% by weight, preferably no more than 10% by weight and very especially preferably no more than 8% by weight of the at least one pharmaceutically active substance selected from the group of cannabinoids has been degraded.

Accordingly, it is preferred that after storage for 2 months at 25° C. and 60% relative humidity (RH) according to ICH standards, 85% by weight, preferably 90% by weight and very especially preferably 92% by weight of the originally contained at least one pharmaceutically active substance selected from the group of cannabinoids is still contained in the oral thin film according to the invention.

The active substance content is determined by means of the HPLC method.

Furthermore, the oral thin film according to the invention may contain additional additives known to a person skilled in the art. These additives include, for example, flavourings, colourants, taste-masking substances, permeation enhancers, sweeteners, fillers, liquid, preferably lipophilic excipients and/or pH stabilisers.

These additives are each preferably present in an amount from about 0.01 to about 10% by weight, in relation to the total weight of the oral thin film. Furthermore, it is preferred that the outer hydrophilic phase of the oral thin film according to the invention is about 30 to about 90% by weight, preferably about 30 to about 80% by weight, in relation to the total weight of the oral thin film.

It is also preferred that the inner hydrophobic phase of the oral thin film according to the invention is about 5 to about 70% by weight, preferably about 10 to about 60% by weight, in relation to the total weight of the oral thin film.

If the specified amount is exceeded or not reached, this has the disadvantage that a homogeneous emulsion can no longer be produced or the necessary amount of active substance can no longer be accommodated in the oral thin film according to the invention.

The oral thin film according to the invention is preferably characterised in that the amount of emulsifier is about 1 to about 15% by weight, preferably about 2 to about 10% by weight, in relation to the total weight of the oral thin film.

If the amount of emulsifier is exceeded, this has the disadvantage that the emulsion changes physically. If the amount of emulsifier is undershot, this has the disadvantage that the hydrophobic phase cannot be stabilised in the hydrophilic phase.

The oral thin film according to the invention preferably has an area of about 0.5 cm² about 10 cm², more preferably from about 2 cm² to about 8 cm².

Preferably, the oral thin film according to the invention has a weight per unit area from about 10 g/m² to about 300 g/m², preferably from about 20 to about 250 g/m², and especially preferably from about 50 g/m² to about 225 g/m².

This preferably corresponds to a layer thickness from about 20 μm to about 500 μm, preferably from about 50 μm to about 300 μm, especially preferably from about 100 to 200 μm, and very especially preferably from about 100 to 150 μm.

Especially preferably, the oral thin film according to the invention is also flexible, which increases wearing comfort in the oral cavity.

This preferred flexibility is especially pronounced in slim oral thin films with a film thickness of about 100 to 200 μm or of about 100 to 150 μm.

It is further preferred that the oral thin film according to the invention is substantially colourless and/or substantially transparent. In an alternative, preferred embodiment, the oral thin film according to the invention is white.

The oral thin film according to the invention dissolves in the oral cavity preferably in a period of less than about 30 min, more preferably in a period of less than about 15 min and very especially preferably in a period of less than about 10 min.

Preferably, the oral thin film according to the invention comprises an outer hydrophilic phase comprising about 50 to about 80% by weight, preferably about 65 to about 70% by weight, of polyvinyl alcohol, an inner hydrophobic phase comprising about 2 to about 20% by weight, preferably about 5 to about 15% by weight, of isopropyl myristate, and about 2 to about 20% by weight, preferably about 5 to about 15% by weight, of vitamin E or a pharmaceutically acceptable derivative thereof, especially a-tocopherol acetate, and about 1 to about 20% by weight, preferably about 5 to about 15% by weight, of at least one active substance selected from the group of cannabinoids, especially tetrahydrocannabinol, the % by weight in each case relating to the total weight of the oral thin film.

The term “comprising” as used here can also mean “consisting of”.

The present invention further relates to a method for producing the oral thin film according to the invention. The method comprises the steps of
a1) producing an aqueous solution or dispersion comprising the at least one hydrophilic polymer;
a2) producing a solution or dispersion comprising at least one pharmaceutically active substance selected from the group of cannabinoids and the at least one hydrophobic substance, wherein at least one of the two solutions of steps a1) or a2) additionally comprises the at least one emulsifier and/or the solution or dispersion of step a2) comprises the vitamin E and/or a pharmaceutically acceptable derivative of vitamin E;
b) mixing the two solutions or dispersions from steps a1) and a2) to obtain an emulsion; and
c) spreading and drying the emulsion obtained in step b) so that the dried emulsion has a weight per unit area of about 50 to about 250 g/m².

Preferably, the method described is carried out under an inert gas atmosphere, preferably a nitrogen atmosphere, or under oxygen-displacing conditions.

The present invention further relates to an oral thin film obtainable by the method described above.

Lastly, the present invention relates to the oral thin films described in more detail above and to the oral thin films obtainable by the method described above as medicaments.

Further, the present invention relates to the oral thin films described in more detail above and to the oral thin films obtainable by the method described above for use in the treatment of pain conditions, nausea and vomiting, neuropathic pain, anorexia, cachexia, multiple sclerosis, traumatic paraplegia, dystonic movement disorders, bronchial asthma, epileptic seizures, withdrawal symptoms of alcohol, benzodiazepine and opiate dependence, Parkinson's disease, dementia, Alzheimer's disease, arthritis, glaucoma, migraine and/or dysmenorrhoea.

The invention also relates to delivery forms for an active substance selected from the group of cannabinoids having a cavity in which the active substance is present, the delivery form being water soluble so that it dissolves rapidly in the mouth upon ingestion and releases the active substance selected from the group of cannabinoids. The fact that the active substance selected from the group of cannabinoids can be introduced into a cavity of the delivery form circumvents technical limitations for the inclusion of larger amounts of active substances in typical thin-film formulations, so that larger amounts of active substance can also be included in the delivery form without difficulty. In addition, the thermal load of active substances in this method is significantly lower compared to conventional production of such delivery systems, which means that a longer storage time can be achieved. The present invention further relates to methods for producing corresponding delivery forms.

For the administration of active substances selected from the group of cannabinoids via the oral mucosa, buccal or sublingual tablets can usually be used, which release the active substance selected from the group of cannabinoids in the oral cavity. The absorption of the active substance via the oral mucosa offers several advantages over other peroral dosage forms, for example the fact that the onset of action is rapid due to bypassing the gastrointestinal passage and that the utilisation of the active substance is high.

Another problem with tablets or capsules is that they are usually swallowed, which requires the patient to have a liquid ready to take this dosage form. Sometimes, however, older patients or children have difficulty swallowing, so that they refuse to take tablets or capsules or are reluctant to take them. In addition, it is possible that patients keep tablets and capsules in their mouths for a longer period of time and then spit them out. This often results in poor compliance, which is detrimental to the progress of healing and the success of the therapy.

As an alternative dosage form to the known buccal and sublingual tablets, planar oblate-like dosage forms are known, which are also referred to as wafers. For example, U.S. Pat. No. 5,529,782 describes a fast-dissolving film product of soluble polymeric material or complex polysaccharides used primarily for the administration of contraceptives. The film product is said to have a thickness of 3 to 4 mm and its solubility is said to be adjustable such that it is dissolved within 5 to 60 seconds after administration. The film product may also be in the form of a laminate having gas-foamed voids.

A carrier material for the administration of medicaments is known from EP 0 450 141 B1, which dissolves quickly on contact with saliva. This carrier material is a porous, dehydrated, skeleton-like carrier material, especially based on proteins and polysaccharides. The cavities created by dehydration are used for the introduction of liquid active substances.

In WO 00/18365, an edible film is proposed that is intended to be rapidly soluble but also to adhere well to the oral mucosa to deliver antimicrobial substances and reduce the number of undesirable microorganisms in the oral flora. The antimicrobial substances are, for example, essential oils, which are mixed as a lipophilic phase, preferably with pullulan as a matrix material in the aqueous phase.

WO 02/02085 describes rapidly disintegrating dosage forms for the release of active substances in the oral cavity or other body orifices, the dosage form having a matrix which contains at least one water-soluble polymer as a basic substance and which is provided with cavities.

Oral thin film (OTF) systems that dissolve rapidly in the oral cavity must also be formulated so that the film meets certain physical requirements. For example, such films must have a certain minimum strength so that they do not break when handled by the patient. Another problem with OTF formulations is that the films cannot be produced in any thickness, as the essential property of the films is that they dissolve rapidly in the mouth. However, this is no longer guaranteed with relatively thick films, as the access of water or saliva to the inner area of the film is more difficult with a higher thickness.

Furthermore, OTF systems are limited not only in terms of their thickness but also in terms of their maximum size, as the user should be able to place the film in the mouth, for example buccally, gingivally and on or under the tongue, without any problems; this would no longer be possible with very large films. Due to these general conditions, the amount of active substance to be applied is limited to about 20 mg for normal OTF formulations in film form.

On the one hand, this is a problem with active substances that have to be applied in higher quantities, but on the other hand, it is also a problem with bitter active substances or other active substances that are perceived as having an unpleasant taste, as these usually have to be formulated with significant amounts of taste-masking agents.

In addition, the active substances from the cannabinoid group especially are relatively susceptible to oxidation and thus relatively unstable when they are coarsely dispersed in a water-soluble polymer. Especially, tetrahydrocannabinol (THC), when embedded in a hydrophilic matrix, is present in waxy/resinous form as a separate phase, which is disadvantageous with regard to oxidative degradation of the active substance and the stability of the oral thin film with regard to phase separation. Due to the sensitivity of the active substances from the group of cannabinoids to oxidative degradation, the oral thin films known from the prior art are disadvantageous, especially with regard to their storage stability.

Against this background, there is a need for a form of administration of active substances that has the same advantages as the known OTF formulations, i.e. especially a rapid dissolution and release of the active substance in the oral cavity, but on the other hand is not subject to such a strong restriction with regard to the possible amount of active substance to be applied.

Another problem with the known OTF systems is that, in order to produce the films, active substances must be mixed with the matrix material used, which is especially difficult to do with the non-water-soluble active substances from the cannabinoid group, for which either a solvent can be used or mixing takes place as part of an extrusion process. When processing with the aid of solvents, this solvent must be removed from the system in the further course of the process, for which purpose the system is usually heated. This poses a problem for temperature labile active substances, such as the active substances selected from the group of cannabinoids, as the active substances integrated in the OTF can decompose/degrade during the evaporation of the solvent. Alternatively, the solvent can also be removed from the solutions under a slight vacuum. However, this requires suitable equipment and, from a technical point of view, can only be realised with greater effort, which entails cost disadvantages.

In an extrusion process, the active substances are also exposed to a higher temperature, which can lead to a partial decomposition of the active substance.

Against this background, there is also a need for a delivery form for an active substance selected from the group of cannabinoids, which can be produced without the active substance selected from the group of cannabinoids having to be exposed to high temperatures. Thus, it is intended that delivery forms can be produced which can also be loaded with temperature-labile active substances. Especially, a delivery form comprising an active substance selected from the group of cannabinoids is to be produced which can be stored for a longer period of time, especially longer than . . . days, without the active substance from the group of cannabinoids being significantly degraded, especially oxidatively.

The present invention addresses this need.

To address the problem described above, the present invention according to claim 15 proposes a delivery form for at least one active substance selected from the group of cannabinoids for dissolving in the oral cavity, comprising a first film layer and a second film layer arranged over the first film layer, wherein the composition of the first film layer can be identical to that of the second layer and comprises a water-soluble polymer, wherein the first and second film layers are joined to one another via their overlapping edges to form at least one cavity, and wherein the cavity is filled with at least one active substance selected from the group of cannabinoids.

Accordingly, the delivery form according to the invention preferably consists substantially of a pocket formed by two film layers arranged one over the other, which pocket is formed by joining the film layers in the edge region. An active substance selected from the group of cannabinoids can then be introduced into the cavity of the pocket. Since the two film layers comprise water-soluble polymers analogous to regular OTF formulations, they have similar dissolution properties as compared to regular OTF formulations. Compared to these, however, the delivery forms according to the invention offer the advantage that the active substance selected from the group of cannabinoids can only be introduced after the films have dried, so that direct thermal stress on the active substance, for example as a result of the drying of the films, is avoided.

The fact that the delivery form is designed as a “water-soluble” pocket means that considerably larger quantities of active substance and/or additional excipients can be introduced into the cavity. A final closure of the pocket after insertion of the active substance can be carried out via only one edge of the delivery form, for which purpose a seal only has to be carried out in the edge region of the delivery form, so that the active substance located in the centre of the delivery form does not have to be exposed to direct thermal stress.

In the context of the present invention, a “water-soluble polymer” means water-soluble and/or water-swellable polymers that rapidly dissolve and disintegrate in a moist and aqueous environment, such as the oral cavity, and thus release an active substance included in the delivery form.

The statement that the first and second film layers “are joined to each other via their overlapping edges to form at least one cavity” is to be understood as meaning that the first and second film layers can touch each other in the region of their surface (provided the cavity is not filled), but are not joined to each other in this region, so that the two film layers can be separated from each other in this region without effort by introducing a material (especially the active substance). The statement also includes round embodiments of the film layers, in which case there is only one overlapping edge but, in order to allow an active substance to be introduced, this edge is not joined over its entire circumference.

The “cavity” contains the active substance selected from the group of cannabinoids but is substantially free of water-soluble polymer.

The joining of the first film layer to the second film layer can be achieved expediently by gluing or sealing. In the case of gluing, for example, a suitable adhesive can be introduced into the intermediate space between the first and second film layers and the first film layer can thus be attached to the second film layer. For sealing, the first film layer and the second film layer can be heated and pressed against each other so that the first film layer adheres to the second film layer in the region of the seal.

It has already been mentioned above that the composition of the first film layer can be identical to that of the second film layer. Since this leads to a simplification of the production of the delivery form according to the invention, it is preferred within the context of the present invention if the composition of the first film layer and the second film layer is identical.

Embodiments are also conceivable in which only one film layer is present, with a cavity being created by folding the film layer and by sealing, into which cavity, among other things, the active substance selected from the group of cannabinoids can be introduced. Such an embodiment has the advantage that a sealing seam can be omitted.

On the other hand, in certain cases it may be expedient if the first film layer and the second film layer are based on different compositions. For example, it may be desirable to form one of the film layers as a mucoadhesive layer, while the second layer is relatively rapidly soluble in an aqueous environment so that the active substance is released. In another embodiment, it may be expedient if the first film layer is formed as a mucoadhesive film layer and the second film layer dissolves more slowly in the oral cavity than the first film layer.

With respect to the form of the active substance selected from the group of cannabinoids, the present invention is not subject to any substantial limitations. Thus, the active substance selected from the group of cannabinoids may be present in liquid form or in the form of a solution or in solid form, with powdered, granular, micro- or nanoparticulate or micro- or nano-encapsulated forms being especially suitable as solid forms. If the active substance is present in liquid form or in the form of a solution, this form should preferably not attack the adjacent film layers where possible. A binding material can also be provided in the cavity and can bind the active substance in liquid form or in the form of a solution. Especially, lactose can be used as a binding material.

As indicated above, the main advantage of the delivery form according to the invention is that it also allows the inclusion of relatively large active-substance-containing fillings. For example, it is preferred if the delivery form has an amount of active-substance-containing filling that is greater than about 20 mg and especially greater than about 30 mg. An especially favourable range for the active-substance-containing filling can be given as an amount of about 50 to about 1000 mg.

The size of the delivery form according to the invention is expediently dimensioned to accommodate a corresponding amount of active-substance-containing filling. As a rough guideline, an area in the range of about 1 to about 10 cm², and preferably about 1.5 to about 6 cm², can be indicated. If the delivery form is, for example, in the form of a rectangular pocket, this may have dimensions of about 2×2.5 cm or about 1×1.5 cm.

The delivery form according to the invention is generally thin and flat or slightly curved, for example in the form of small pockets, bags, sachets or pads. These small pockets, bags, sachets or pads may be of various geometric shapes, for example circular, elliptical, elongate or angular, such as especially rectangular or square.

The thickness of the film layers is preferably about 0.01 to about 2 mm, especially preferably it is in the range of about 0.02 to about 0.5 mm.

With respect to the water-soluble polymer, the present invention is not subject to any relevant limitations, with the proviso that the water-soluble polymer should be a pharmaceutically acceptable material. Suitable water-soluble polymers are, for example, starch and starch derivatives, dextrans; cellulose derivatives, such as carboxymethyl cellulose, hydroxypropyl cellulose, hydroxyethyl cellulose, hydroxypropylmethyl cellulose, hydroxypropyl ethyl cellulose, sodium carboxymethyl cellulose, ethyl or propyl cellulose; polyacrylic acid, polyacrylates, polyvinylpyrrolidones, polyvinyl alcohol, polyethylene oxide polymers, polyacrylamides, polyethylene glycol, gelatine, collagen, alginates, pectins, pullulan, tragacanth, chitosan, alginic acid, arabinogalactan, galactomannan, agar, agarose, carrageenan and natural gums.

Especially preferred in the context of the present invention are water-soluble polymers selected from the group comprising polyvinyl alcohol, polyethylene glycol, polyethylene oxide, cellulose derivatives, pullulan, gelatine and agar. Most preferred in the context of the present invention is polyvinyl alcohol as a water soluble polymer.

The proportion of water-soluble polymer in the first and second film layers is usually about 50 to about 100% by weight, especially about 90 to about 99.9% by weight and most preferably about 90 to about 99.5% by weight. Since, in contrast to classic OTF systems, the polymer film does not have to contain an active substance, the proportion of water-soluble polymer can be very high. On the other hand, depending on the intended application result, additives such as taste-masking agents or part of the active substance and/or another, different pharmaceutically active substance may be included in the first film layer and/or in the second film layer. In this case, the amount of water-soluble polymer in the first film layer and in the second film layer may be less than indicated above, but should still be in the range of about 15 to about 75% by weight, and preferably about 50 to about 70% by weight.

The active substance is selected here from the group of cannabinoids.

The term cannabinoids is a collective term for terpenoid constituents with 21 carbon atoms that can be isolated from cannabis species, mainly benzopyran derivatives, and their (semi-)synthetic derivatives. More than 70 naturally occurring cannabinoids are known, some of which have psychotropic and other pharmacological effects.

A non-exhaustive list of examples of active substances from the group of cannabinoids comprises cannabichromanone, cannabichromene, cannabicoumaronone, cannabicyclol, cannabidiol, cannabidivarin, cannabidivaric acid, cannabifuran, cannabinodiol, cannabinol, cannabinolic acid, cannabitriol, cannabivarichromene, cannabivarin, Δ8-tetrahydrocannabinol, Δ9-tetrahydrocannabinol.

The active substances from the group of cannabinoids can be of natural, semi-synthetic or synthetic origin.

In addition, R-(6a, 10a)-Δ9-tetrahydrocannabinol is suitable as a synthetically produced cannabinoid for administration in the oral thin film according to the invention.

Cannabis extracts and Cannabis oils, especially extracts and oils of Cannabis sativa or Cannabis indica, may also be considered. Cannabis extracts or oils contain tetrahydrocannabinol (predominantly Δ9-tetrahydrocannabinol, in smaller amounts Δ8-tetrahydrocannabinol), cannabidiol, cannabinol and cannabichromene as pharmacologically active ingredients.

Especially preferably, the at least one pharmaceutically active substance selected from the group of cannabinoids is tetrahydrocannabinol (THC), preferably Δ8-tetrahydrocannabinol, Δ9-tetrahydrocannabinol or R-(6a, 10a)-Δ9-tetrahydrocannabinol, cannabinol, cannabidiol, cannabichromene, and/or . . . .

For such active substances, it has been described in WO 03/105800 A2 that they can be included in regular OTF film formulations, thus eliminating the disadvantages of conventional methods of administration, such as capsules, tablets, pills or orally administered liquid preparations.

In addition to the aforementioned water-soluble polymer as a component of the first and second film layers and an active substance selected from the group of cannabinoids, which is located in the cavity between the first and second film layers, the delivery form according to the invention may contain further ingredients, especially excipients selected from the group comprising colourants, aromatic substances, especially flavour and/or odour substances, sweeteners, taste-masking agents, surfactants, enhancers, pH regulators, preservatives, carriers or binders for liquid fillings, such as lactose, and/or antioxidants. The excipients mentioned can be part of one or both film layers and/or can be introduced together with the active substance into the cavity between the two film layers.

The addition of flavourings, odour and aromatic substances, individually or in combination, is especially advantageous. For example, the addition of a flavouring (for example menthol, eucalyptol) can improve the taste impression. At the same time, this enables an inconspicuous ingestion of an active pharmaceutical substance selected from the group of cannabinoids, since the ingested delivery form smells like an ordinary refreshment sweet. This contributes to an improvement in compliance.

A taste-masking agent may be present as a component of both the first and/or second film layers, but it may also be introduced into the cavity of the delivery form according to the invention, as is evident above.

If the delivery form according to the invention contains a taste-masking agent, this can be included in one film layer or both film layers, or, in the case of a multi-layer film structure, in one outer layer or the multiple outer layers of the films. In this way, for example, an early release of the taste-masking agent from an outer polymer layer compared to the release of the active substance can be realised, so that the taste receptors, for example against bitter-tasting active substances inside the delivery form according to the invention, can already be saturated before the active substance is released.

In addition, the first and/or the second film layer may contain at least one pigment or UV-absorbing agent that protects a photosensitive active substance introduced into the cavity of the delivery form from UV light. In addition, it is expedient if the first and/or the second film layer contain one or more colourants, flavourings or sweeteners.

In addition to the excipients already mentioned above, the first film layer and/or the second film layer may also contain further components to optimise their flexibility or other physical properties, such as at least one plasticiser and/or a humectant. Preferred plasticisers and/or humectants in the context of the present invention are selected from the group comprising glycerol, propylene glycol, polyethylene glycol and citric acid esters.

Furthermore, the first film layer and/or the second film layer can be designed as a foam, i.e. can contain an introduced gas, such as air, nitrogen or CO₂, or another gas.

As indicated above, the first film layer and the second film layer in the delivery form according to the invention may be single-layered or multi-layered, it also being possible that the first film layer and/or the second film layer is built up of a plurality of layers of the same composition, for example by producing the first film layer or the second film layer by applying the composition in layers on top of each other. On the other hand, the layers can differ in their composition, for example by introducing a pigment or a UV-absorbing agent into a layer of the first film layer or second film layer and overlaying or underlaying it with a composition without pigments or UV-absorbing agent.

In a multi-layer structure, one or more of the layers can be embodied as a foam, i.e. can contain an introduced gas, such as air, nitrogen or CO₂, or another gas.

In addition, the delivery form according to the invention can also be designed in such a way that it has two cavities that are spatially separated from each other. For example, the first film layer can be joined to the second film layer via an additional seal in the region of its surface. This allows two or more cavities to be formed between the film layers, which cavities can have the same or different fillings.

To increase the volume present between the two film layers of the delivery form according to the invention, it is also possible for the first film layer and/or the second film layer to have a non-planar form. For this purpose, the first film layer or the second film layer can preferably be thermoformed in order to obtain more filling volume with the same base area. Furthermore, it is possible that the delivery form according to the invention also contains active substance introduced into the film layers in addition to the active substance selected from the group of cannabinoids introduced into the cavity. Lastly, it is possible that the active substance selected from the group of cannabinoids introduced into the cavity is introduced in different modifications, for example one part in a direct-release form, while another part is introduced in granulated form or in a sustained-release form, in order to realise mixed kinetics of the release of the active substance selected from the group of cannabinoids.

Due to the properties already mentioned, the delivery forms according to the invention containing cannabinoids as active substances can be advantageously used in the treatment of diseases or symptoms of diseases, especially for use in the treatment of pain conditions, nausea and vomiting, neuropathic pain, anorexia, cachexia, multiple sclerosis, traumatic paraplegia, dystonic movement disorders, bronchial asthma, epileptic seizures, withdrawal symptoms of alcohol, benzodiazepine and opiate dependence, Parkinson's disease, dementia, Alzheimer's disease, arthritis, glaucoma, migraine and/or dysmenorrhoea.

Accordingly, an aspect of the present invention also relates to a delivery form of the type described above, containing a cannabinoid as active substance, for the treatment of at least one of the aforementioned diseases.

In this case, the delivery form can be expediently applied via the oral mucosa, especially sublingually or buccally.

Lastly, another aspect of the present invention relates to a method for producing a delivery form of the type described above, the method comprising the following steps of:
a) positioning a first and a second film layer on top of each other,
b) attaching the first film layer to the second film layer in a manner such that at least one pocket is formed between the first film layer and the second film layer,
c) if necessary, cutting the film bilayers obtained in b) while retaining individual pockets,
d) filling the at least one pocket with at least one active substance selected from the group of cannabinoids and possible excipients, and
e) closing the pocket(s).

The attachment in step b) and/or the closing of the pocket (in step e)) is preferably done by gluing or sealing as part of this process.

Positioning a first film layer and a second film layer on top of each other in step a) can be done either by positioning two individual films on top of each other or by bending a film in its middle so that two film layers arranged one over the other are formed and are joined together at one edge.

The invention is explained below by means of non-limiting examples.

### EXAMPLE 1

TABLE 1

| | | Quantity |
|---|---|---|
| | Substance | [% by weight] |
| Composition | polyvinyl alcohol | 70.0 |
| suitable for oral | α-tocopherol acetate | 10.0 |
| thin films | isopropyl myristate | 10.0 |
| | tetrahydrocannabinol | 10.0 |

Oral thin films with the composition listed in Table 1 with 5.03 mg dronabinol (THC) as active substance and a size of 4.11 cm² were produced in accordance with the method according to the invention and subsequently tested for stability.

For this purpose, the oral thin films were stored at 5° C., at 25° C. and 60% RH, and at 40° C. and 75% RH (in each case according to ICH standards from 2003) for 2 months (ICH standards are understood here to be the publication “Guidance for Industry Q1A(R2) Stability Testing of New Drug Substances and Products” of the U.S. Department of Health and Human Services, Food and Drug Administration, Center for Drug Evaluation and Research (CDER), Center for Biologics Evaluation and Research (CBER), November 2003, ICH, Revision 2).

Subsequently, the remaining dronabinol content and the degradation products of dronabinol were determined by means of the HPLC method. In each case, 3 independent tests were carried out and the mean value determined.

The results of the stability test are summarised in Tables 2 to 4.

TABLE 2

| | Content of dronabinol after 2 months of storage | | |
|---|---|---|---|
| Initial | at | | |
| dronabinol | | 25° C. and 60% | 40° C. and 75% |
| content | 5° C. | RH | RH |
| 5.3 mg | 4.97 ± 0.038 mg | 4.89 ± 0.102 mg | 4.99 ± 0.02 mg |
| 100% | 93.8 ± 0.8% | 92.3 ± 1.93% | 94.2 ± 0.38% |

TABLE 3

| | Content of dronabinol after 6 months of storage | | |
|---|---|---|---|
| Initial | at | | |
| dronabinol | | 25° C. and 60% | 40° C. and 75% |
| content | 5° C. | RH | RH |
| 5.3 mg | 4.80 ± 0.28 mg | 4.80 ± 0.2 mg | 4.90 ± 0.1 mg |
| 100% | 90.6 ± 5.28% | 90.6 ± 3.77% | 92.5 ± 1.89% |

TABLE 4

| | Degradation products of dronabinol | | |
|---|---|---|---|
| Storage conditions | Initial | After 2 months | After 6 months |
| 5° C. | 0.67% | 0.70% | 0.77% |
| 25° C. and 60% RH | | 1.06% | 1.22% |
| 40° C. and 75% RH | | 1.42% | 1.58% |

The stability studies show a high stability of the active substance dronabinol in the oral thin films according to the invention.

### EXAMPLE 2

Polymer films with the compositions indicated in Table 6 were formulated and provided with a filling as indicated in Table 6. For this purpose, the various polymer films were first cast from solutions of the stated ingredients, which were dried to form a film. Then, corresponding film pieces with the dimensions indicated in Table 1 were punched out, placed on top of each other to form a double layer, and joined together at three of the edges of the film by heat sealing. The filling was then poured in and the resulting pocket was also sealed at the open edge likewise by heat sealing.

TABLE 5

| | A | B | C | D |
|---|---|---|---|---|
| Polymer film | | | | |
| Polyvinyl alcohol | 96.9% | | | 100% |
| Polyol N10 | | 95.9% | | |
| Pullulan | | | 95.6% | |
| Xanthan | | | 0.3% | |
| Colourant | 0.1% | 0.1% | 0.1% | |
| Flavouring | 1% | 1% | 1% | |
| Sweetener | 2% | 3% | 3% | |
| Weight per unit | 45 g/m² | 45 g/m² | 45 g/m² | 50 g/m² |
| area | | | | |
| Polymer film 2 | like | like | like | like |
| | polymer | polymer | polymer | polymer |
| | film 1 | film 1 | film 1 | film 1 |
| Filling | | | | |
| THC | 12.5% | 12.5% | 12.5% | 7.7% |
| Lactose | 83.5% | 83.5% | | 76.9% |
| Hard fat or cocoa | | | 83.5% | |
| butter | | | | |
| Flavouring | 1% | 1% | 1% | |
| Sweetener | 3% | 3% | 3% | |
| Vitamin E | | | | 7.7% |
| Isopropyl | | | | 7.7% |
| myristate | | | | |
| Filling quantity | 20 − 80 mg | 20 − 80 mg | 20 − 80 mg | 20 − 150 mg |
| Pocket size | 20 × 25 mm | 20 × 25 mm | 20 × 25 mm | 20 × 25 mm |

For formulation D, the remaining dronabinol content and the degradation products of dronabinol were determined using the HPLC method. In each case, 3 independent tests were carried out and the mean value determined. The results of the stability test are summarised in Tables 6 and 7.

TABLE 6

| Initial | Content of dronabinol after 2 months of storage at | | |
|---|---|---|---|
| dronabinol | | 25° C. and 60% | 40° C. and 75% |
| content | 5° C. | RH | RH |
| 8.56 mg | 8.03 ± 1.29 mg | 7.97 ± 0.96 mg | 8.44 ± 0.38 mg |
| 100% | 93.8 ± 15.07% | 90.6 ± 11.21% | 92.5 ± 4.44% |

TABLE 4

| | Degradation products of | |
|---|---|---|
| | dronabinol | |
| Storage conditions | Initial | After 2 months |
| 5° C. | 2.47% | 9.76% |
| 25° C. and 60% RH | | 8.31% |
| 40° C. and 75% RH | | 4.47% |

The stability studies show a high stability of the active substance dronabinol in the oral thin films according to the invention.

1. An oral thin film comprising an outer hydrophilic phase, which contains at least one hydrophilic polymer, and an inner hydrophobic phase, which contains at least one hydrophobic substance, and at least one pharmaceutically active substance selected from the group of cannabinoids, wherein the oral thin film additionally comprises at least one emulsifier and/or vitamin E and/or a pharmaceutically acceptable derivative of vitamin E.

2. The oral thin film according to claim 1, characterised in that the at least one pharmaceutically active substance selected from the group of cannabinoids is present substantially in the inner hydrophobic phase.

3. The oral thin film according to claim 1, characterised in that the at least one pharmaceutically active substance selected from the group of cannabinoids is tetrahydrocannabinol.

4. The oral thin film according to claim 1, characterised in that the amount of the at least one pharmaceutically active substance selected from the group of cannabinoids is about 1 to about 30% by weight in relation to the total weight of the oral thin film.

5. The oral thin film according to claim 1, characterised in that the at least one hydrophilic polymer in the outer hydrophilic phase is selected from the group consisting of starch and starch derivatives, dextran, cellulose and cellulose derivatives, carboxymethyl cellulose, hydroxypropyl cellulose, hydroxyethyl cellulose, hydroxypropylmethyl cellulose, hydroxypropyl ethyl cellulose, sodium carboxymethyl cellulose, ethyl or propyl cellulose, polyacryllc acid, polyacrylate, polyvinyl pyrrolidone, polyvinyl alcohol, polyethylene oxide polymers, polyacrylamide, polyethylene glycol, gelatine, collagen, alginate, pectin, pullulan, tragacanth, chitosan, alginic acid, arabinogalactan, galactomannan, agar-agar, agarose, carrageenan, natural gums and/or copolymers thereof.

6. The oral thin film according to claim 1, characterised in that the at least one hydrophobic substance in the inner hydrophobic phase comprises medium-chain triglycerides, fatty acids, especially isopropyl myristate and/or mixtures thereof.

7. The oral thin film according to claim 1, characterised in that the at least one emulsifier comprises polysorbate, sorbitan esters, polyoxyethylene fatty acid ethers, macrogol glycerol hydroxy stearates, glycerol mono- and dibleates and/or mixtures thereof.

8. The oral thin film according to claim 1, characterised in that vitamin E and/or the pharmaceutically acceptable derivative of vitamin E is present in an amount from about 1 to about 30% by weight in relation to the total weight of the oral thin film.

9. The oral thin film according to claim 1, characterised in that the outer hydrophilic phase constitutes 30 to 80% by weight in relation to the total weight of the oral thin film.

10. The oral thin film according to claim 1, characterised in that the inner hydrophobic phase constitutes about 10 to about 60% by weight in relation to the total weight of the oral thin film.

11. The oral thin film according to claim 1, characterised in that the amount of emulsifier is about 2 to about 10% by weight in relation to the total weight of the oral thin film.

12. The oral thin film according to claim 1, characterised in that, after storage for 2 months at 25° C. and 60% relative humidity, at least 85% by weight of the originally contained at least one pharmaceutically active substance selected from the group of cannabinoids is still contained in the oral thin film according to the invention,

13. A method for producing the oral thin film of claim 1, comprising the steps of:
a1) producing an aqueous solution or dispersion comprising the at least one hydrophilic polymer;
a2) producing a solution or dispersion comprising at least one pharmaceutically active substance selected from the group of cannabinoids and the at least one hydrophobic substance, wherein at least one of the two solutions or dispersions of steps a1) or a2) additionally comprises the at least one emulsifier and/or the solution or dispersion of step a2) comprises the vitamin E and/or a pharmaceutically acceptable derivative of vitamin E;
b) mixing the two solutions or dispersions from steps a1) and a2) to obtain an emulsion; and
c) spreading and drying the emulsion obtained in step b) so that the dried emulsion has a weight per unit area of about 20 to 250 g/m².

14. An oral thin film obtained by the method according to claim 13.

15. A method for the treatment of pain conditions, nausea and vomiting, neuropathic pain, anorexia, cachexia, multiple sclerosis, traumatic paraplegia, dystonic movement disorders, bronchial asthma, epileptic seizures, withdrawal symptoms of alcohol, benzodiazepine and opiate dependence, Parkinson's disease, dementia, Alzheimer's disease, arthritis, glaucoma, migraine, or dysmenorrhoea comprising the administration of an effective amount of the oral thin film of claim 1.

16. A delivery form for at least one active substance selected from the group of cannabinoids for dissolving in the oral cavity, comprising a first film layer and a second film layer arranged over the first film layer, wherein the composition of the first film layer can be identical to that of the second layer and comprises a water-soluble polymer, wherein the first and second film layers are joined to one another via their overlapping edges to form at least one cavity, and wherein the cavity is filled with at least one active substance selected from the group of cannabinoids.

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. A method for producing a delivery form according to claim 16 comprising the steps of:
a) positioning a first film layer and a second film layer on top of each other,
b) attaching the first film layer to the second film layer in such a manner that at least one pocket is formed between the first film layer and the second film layer,
c) if necessary, cutting the film bilayers obtained in b) while retaining individual pockets,
d) filling the at least one pocket with at least one active substance selected from the group of cannabinoids, and possible excipients,
e) closing the pocket(s).

32. (canceled)

33. The oral thin film according to claim 1, characterised in that the at least one pharmaceutically active substance selected from the group of cannabinoids is Δ8-tetrahydrocannabinol, Δ9-tetrahydrocannabinol or R-(6a, 10a)-Δ9-tetrahydrocannabinol, cannabinol, cannabidiol, and/or cannabichromene.

34. The oral thin film according to claim 1, characterised in that the amount of the at least one pharmaceutically active substance selected from the group of cannabinoids is about 5 to about 20% by weight in relation to the total weight of the oral thin film.

35. The oral thin film according to claim 1, characterised in that vitamin E and/or the pharmaceutically acceptable derivative of vitamin E is present in an amount from about 5 to about 20% by weight in relation to the total weight of the oral thin film.
